(19) United States
(12) Patent Application Publication
(10) Pub. No.: US 2022/0404341 A1
(43) Pub. Date: Dec. 22, 2022

(54) METHODS AND APPARATUS FOR MEASURING IMMUNE MEDIATED TUMOROID RESPONSES

(71) Applicant: VITROSCAN B.V., Leiden (NL)

(72) Inventors: Willemijn VADER, Leiden (NL); Abbas JARIANI, Leiden (NL); Leo PRICE, Leiden (NL); Fanny GRILLET, Leiden (NL)

(21) Appl. No.: 17/771,431

(22) PCT Filed: Oct. 23, 2020
(86) PCT No.: PCT/NL2020/050660

§ 371 Date: Apr. 22, 2022

(30) Foreign Application Priority Data
Oct. 26, 2019 (NL) 2024108

(51) Int. Cl. G01N 33/50

(52) U.S. Cl. CPC G01N 33/5082; G01N 33/5011

(57) ABSTRACT

The present invention relates to a method for measuring the immune-mediated effect of one or more immunotherapeutic agents on patient derived tumour cultures, using 3-dimensional visualisation of the tumour cell cultures. It also relates to the tumour cell cultures, and to a kit of parts comprising cell cultures and apparatus.

### FIELD OF THE INVENTION

The present invention relates to a method for measuring the immune-mediated effect of one or more immunotherapeutic agents on patient derived tumour cultures. Specifically, the invention provides an image-based algorithm to analyse responsiveness of fresh and cryopreserved tumour cell aggregates, and thus enables to pre-select patients that are responsive to treatments.

### BACKGROUND OF THE INVENTION

Cancer immunotherapy involves the attack of cancer cells by a patient's immune system. Regulation and activation of immune cells such as T-lymphocytes depends on signalling by the T cell receptor and also co-signalling receptors that deliver positive or negative signals for activation. Immune responses by T-cells are thus controlled by a balance of costimulatory and inhibitory signals, called immune checkpoints. Immuno-modulation therapy, which attends to this process, more specifically immunotherapeutic agents such as immune checkpoint modulators, has been revolutionizing cancer therapy. For example, in certain melanoma patients, anti-CTLA4 and anti-PD1 antibodies have offered a remarkable opportunity for long-term disease control in the metastatic setting. However, not all patients, even where biomarkers may indicate susceptibility, are responding positively to the therapy. Accordingly, given the high cost, significant side effects, and patient-to-patient response variability of immunotherapeutic agents for cancer treatment, it would be highly appealing to develop an in-vitro assay to predict response of individual patients to these treatments, from naïve patient samples. Applicants have now surprisingly found that by creating a cell culture from naïve samples, and by subjecting it to a test treatment with immunotherapeutic agents, an effective patient selection can be made that allows to target only those patients who respond to the treatment, i.e. whereby the individual patient immune cells and tumour cells show susceptibility to the treatment.

### SUMMARY OF THE INVENTION

The present invention encompasses the discovery that the likelihood of a favourable response to cancer immunomodulation therapy can be predicted. The present invention further comprises the discovery that a cell culture comprising both a patient's immune cells and tumour cells permits to predict which cancer patients are likely to respond to immunotherapy, in particular, treatment with an immune checkpoint modulator.

Accordingly, in a first aspect, the present invention relates to a method for measuring the immune-mediated effect of one or more immunotherapeutic agents on patient derived tumour cultures, the method comprising: (a) preparing a three-dimensional size-normalised culture in a multitude of replicates comprising patient derived tumour cell aggregates in a growth medium; (b) adding the one or more immunotherapeutic agents to the culture, and (c) culturing for a pre-defined time period; and (d) determining the effect that the one or more immunotherapeutic agents has on the tumour cell aggregates by measuring the total area of objects in the culture that are above about 420 μm², and the total area of objects that are below about 160 μm², using three-dimensional imaging of the cell culture, and (e) identifying the patients that are responsive to one or more immunotherapeutic agents. In a second aspect, the present invention also relates to a 3-dimensional cell culture obtainable according to the above method.

In a further aspect, the present invention also relates to a kit comprising the cell culture according to the invention and an imaging analysing apparatus.

In a third aspect, the present invention relates to an in-vitro ovarian and mesothelioma tumour cell culture, comprising three-dimensional cell aggregates.

In a further aspect, the present invention relates to an in-vitro lung tumour cell culture, comprising three-dimensional cell aggregates.

In a further aspect, the present invention relates to an in-vitro ovarian, lung and mesothelioma tumour cell culture, comprising three-dimensional cell aggregates.

In a further aspect, the present invention relates to a test method for testing patient specific drug efficacy.

In yet a further aspect, the present invention relates to ipilimumab for use in the treatment of a patient that has shown a selective response to ipilimumab in the in vitro test method.

In yet a further aspect, the present invention relates to ADU-S100 for use in the treatment of a patient that has shown a selective response to ADU-S100 in the in vitro test method.

In yet a further aspect, the present invention relates to pembrolizumab for use in the treatment of a patient that has shown a selective response to pembrolizumab in the in vitro test method.

In yet a further aspect, the present invention relates to nivolumab for use in the treatment of a patient that has shown a selective response to nivolumab in the in vitro test method.

In yet a further aspect, the present invention relates to combinations comprising ipilimumab for use in the treatment of a patient that has shown a selective response to ipilimumab in the in vitro test method.

In yet a further aspect, the present invention relates to combinations comprising ADU-S100 for use in the treatment of a patient that has shown a selective response to ADU-S100 in the in vitro test method.

In yet a further aspect, the present invention relates to combinations comprising pembrolizumab for use in the treatment of a patient that has shown a selective response to pembrolizumab in the in vitro test method.

In yet a further aspect, the present invention relates to combinations comprising nivolumab for use in the treatment of a patient that has shown a selective response to nivolumab in the in vitro test method.

In another aspect, the present invention relates to a method for identifying agents having patient specific anticancer activity against ovarian cancer and mesothelioma cells comprising selecting at least one test agent, contacting a plurality of ex vivo patient derived ovarian cancer or mesothelioma cell aggregates with the one or more test agents, determining the number, size and viability of tumour cell aggregates in the presence and absence of the test agent, and identifying an agent having anticancer activity if the number, size and viability of aggregates with a size above 420 μm² is lower than in the presence of the agent than in the absence of the agent.

In another aspect, the present invention relates to a method for identifying agents having patient specific anticancer activity against ovarian cancer, lung cancer and mesothelioma cells comprising selecting at least one test agent, contacting a plurality of ex vivo patient derived ovarian cancer or mesothelioma cell aggregates with the one or more test agents, determining the number, size and viability of tumour cell aggregates in the presence and absence of the test agent, and identifying an agent having anticancer activity if the number, size and viability of aggregates with a size above 420 μm² is lower than in the presence of the agent than in the absence of the agent.

In another aspect, the present invention relates to a method for identifying agents having patient specific anticancer activity against lung cancer comprising selecting at least one test agent, contacting a plurality of ex vivo patient derived lung cancer aggregates with the one or more test agents, determining the number, size and viability of tumour cell aggregates in the presence and absence of the test agent, and identifying an agent having anticancer activity if the number, size and viability of aggregates with a size above 420 μm² is lower than in the presence of the agent than in the absence of the agent.

In some aspects, the invention provides methods for treating a patient or subject with an immune checkpoint modulator wherein the subject is identified to have a tumour, wherein the presence of the patient's immune cells may permit application of successful therapy with an immune checkpoint modulator, comprising a step of selecting for receipt of the therapy a subject identified as having a tumour and immune cells susceptible to treatment with administered immune checkpoint modulators, wherein an improvement comprises administering therapy to a subject identified as having a cancer. In some embodiments, the invention provides methods for treating a cancer selected from the group consisting of carcinoma, sarcoma, myeloma, leukaemia, or lymphoma, the methods comprising a step of administering immune checkpoint modulator therapy to a subject identified as having a cancer and immune system susceptible to treatment with administered immune checkpoint modulators.

In an aspect, the invention relates to a method for measuring the immune-mediated effect of one or more immunotherapeutic agents on patient derived tumour cultures, the method comprising: (a) preparing a three-dimensional size-normalised culture in a multitude of replicates comprising patient derived tumour cell aggregates in a growth medium; (b) adding the one or more immunotherapeutic agents to the culture, and (c) culturing for a pre-defined time period; and (d) determining the effect that the one or more immunotherapeutic agents has on the tumour cell aggregates by measuring the total area of objects in the culture that are above about 420 μm², and the total area of objects that are below about 160 μm², using three-dimensional imaging of the cell culture.

### SHORT DESCRIPTION OF THE DRAWINGS

The following figures are presented for the purpose of illustration only, and are not intended to be limiting:

FIG. 1 shows samples where Ipilimumab appears to be effective in activating native immune cells to kill ovarian cancer tumoroids. Plots in the top row indicate total area from objects larger than 422 μm². This metric indicates the abundance and survival of tumour cell clusters. The plots on the bottom row indicate the total area of objects smaller than 160 μm². This metric indicates abundance of immune cells. The indicated p-values correspond to one-sided Wilcoxon test.

FIG. 2—Similar to FIG. 1, but for the ovarian cancer samples where Ipilimumab does not show effectiveness in activating native immune cells to kill tumoroids.

FIG. 3—Similar to FIG. 1, but for a Mesothelioma tumour sample showing immune-mediated tumoroid killing effect when treated with Ipilimumab.

FIG. 4—Similar to FIG. 1, but for an ovarian tumour sample showing immune-mediated tumoroid killing effect when treated with ADU-S100, and pembrolizumab, and combinations thereof.

FIG. 5—Similar to FIG. 1 and same sample as FIG. 4, but for an ovarian tumour sample showing immune-mediated tumoroid killing effect when treated with ADU-S100, and pembrolizumab, and combinations thereof.

FIG. 6—Similar to FIG. 1, but for a lung tumour sample showing immune-mediated tumoroid killing effect when treated with ADU-S100, pembrolizumab, ipilimumab, and nivolumab, and combinations thereof.

FIG. 7—IFN-γ production is increased in conditions treated with Staphylococcal enterotoxin A (SEA, (positive control) and Ipilimumab. IFN-γ is secreted by activated T-cells.

FIG. 8—Tumoroids' area decrease (%) as Selection Factor. This metric is conditionally dependent on statistical increase in number (total area) of small objects due to Ipilimumab treatment, and decrease in area of the tumoroids (large objects) as detailed in Formula I. The filled circle symbols represent the metric for Ipilimumab treatment, and the plus symbols represent the metric for SEA treatment (positive control). In some experiments, Ipilimumab was tested in combination with Nivolumab or Pembrolizumab. In the shown experiments Nivolumab and Pembrolizumab do not show any effect when used alone, but in this figure, we show their combination with Ipilimumab as a biological replicate for Ipilimumab effect, in order to indicate the robustness of this metric.

FIG. 9—Tumoroid's area decrease (%) as Selection Factor. This metric is conditionally dependent on statistical increase in number (total area) of small objects due to treatment, and decrease in area of the tumoroids (large objects) as detailed in Formula I. Each row is a distinct treatment (monotherapy or combination) and each column indicates a sample. All samples are ovarian ascites unless indicated otherwise; suffix “_T” indicates a solid tumour sample and suffixes “_I” and _II” indicate the first and second sample from the same patient, respectively. The shade of grey and number in each cell indicate the selection factor value. Hatched cells indicate that treatment was not tested on the sample.

### DETAILED DESCRIPTION OF THE INVENTION

It must also be noted that as used herein and in the appended claims, the singular forms “a”, “an”, and “the” include plural reference unless the context clearly dictates otherwise. Thus, for example, reference to a “cell” is a reference to one or more cell and equivalents thereof known to those skilled in the art, and so forth. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

As used herein, the term “about” means plus or minus 10% of the numerical value of the number with which it is being used. Therefore, about 50% means in the range of 45%-55%.

“Optional” or “optionally” means that the subsequently described event or circumstance may or may not occur, and that the description includes instances where the event occurs and instances where it does not.

“Administering” when used in conjunction with a therapeutic means to administer a therapeutic to a patient whereby the therapeutic positively impacts the tissue to which it is targeted. Thus, as used herein, the term “administering”, when used in conjunction with cancer therapy, can include, but is not limited to, providing a treatment into or onto the target tissue; providing a treatment systemically to a patient by, e.g., intravenous injection whereby the therapeutic reaches the target tissue; providing a treatment in the form of silencing expression of a specific gene. “Administering” a composition may be accomplished orally, by injection, topical administration, or by these methods in combination with other known techniques.

The term “tissue” refers to any aggregation of similarly specialized cells which are united in the performance of a particular function.

As used herein the term “modulator” means any active agent that modulates the activation state of immune cells, thereby modulating an immune response such as cell killing by effector cells, such as cytotoxic T-cells) in a subject (e.g., a human subject).

As used herein, the term “therapeutic” means an agent utilized to treat, combat, ameliorate, prevent or improve an unwanted condition or disease of a patient. Embodiments of the present invention are directed to regulating cancer cell growth.

The methods and devices of the present invention do not require staining of cells with toxic dyes, and therefore, allows for observation of cell growth or inhibition of cell growth in real time.

The one or more immunotherapeutic agents may be added to the culture in combination with other known anti-cancer therapies or compounds. Preferably, the anti-cancer compound is olaparib.

The term “Ipilimumab” relates to ipilimumab (Yervoy), a monoclonal antibody that targets the protein receptor cytotoxic T-lymphocyte-associated protein 4 (CTLA-4). A preferred ipilimumab is the human monoclonal antibody 10D1 (also referred to as MDX-010 and ipilimumab and available from Medarex, Inc., Bloomsbury, N.J., USA) is disclosed for instance in WO 01/14424.

As noted elsewhere herein, the administration of Ipilimumab and one or more other active anti-CTLA4 antagonists may be administered either alone or in combination with known anti-cancer therapies. Advantageously, this one or more immunotherapeutic agents according to the invention comprises Ipilimumab as a monotherapy or in combination with other immunomodulators. Preferably, the one or more other immunotherapeutic agents is selected from the group consisting of: pembrolizumab and nivolumab. Preferably, the one or more other immunotherapeutic agents is selected from the group consisting of: pembrolizumab, ADU-S100 and nivolumab.

The term “pembrolizumab” relates to pembrolizumab (Keytruda), a monoclonal antibody that targets the protein receptor programmed cell death protein 1 (PD-1), available from Merck, Kenilworth, N.J., USA) is disclosed for instance in EP2170959A1, and in U.S. Pat. Nos. 8,354,509 and 8,900,587.

As noted elsewhere herein, the administration of pembrolizumab and one or more other PD-1 inhibitors may be administered either alone or in combination with known anti-cancer therapies. Advantageously, this one or more immunotherapeutic agents according to the invention comprises pembrolizumab as a monotherapy or in combination with other immunomodulators. Preferably, the one or more other immunotherapeutic agents is selected from the group consisting of: ipilimumab, nivolumab and ADU-S100.

The term “nivolumab” relates to nivolumab (Opdivo), a monoclonal antibody that targets the protein receptor programmed cell death protein 1 (PD-1), and available from Bristol Myers Squibb, New York City, N.Y., USA, and described for instance in U.S. Pat. No. 8,008,449, EP1576014B1.

As noted elsewhere herein, the administration of nivolumab and one or more other PD-1 inhibitors may be administered either alone or in combination with known anti-cancer therapies. Advantageously, this one or more immunotherapeutic agents according to the invention comprises nivolumab as a monotherapy or in combination with other immunomodulators. Preferably, the one or more other immunotherapeutic agents is selected from the group consisting of: pembrolizumab, ipilimumab and ADU-S100.

The term “ADU-S100” relates to ADU-S100 (also referred to as MIW815, available from Chinook Therapeutics, Vancouver, Canada), a synthetic cyclic dinucleotide that targets the Stimulator of Interferon Genes (STING).

As noted elsewhere herein, the administration of ADU-S100 and one or more other active STING agonists may be administered either alone or in combination with known anti-cancer therapies. Advantageously, this one or more immunotherapeutic agents according to the invention comprises ADU-S100 as a monotherapy or in combination with other immunomodulators. Preferably, the one or more other immunotherapeutic agents is selected from the group consisting of: pembrolizumab, nivolumab and ipilimumab.

Advantageously, the one or more immunotherapeutic agents according to the invention may comprise ipilimumab, nivolumab, pembrolizumab or ADU-S100 as a monotherapy or in combination with other immunomodulators. Advantageously, the one or more immunotherapeutic agents according to the invention may comprise ipilimumab, nivolumab or pembrolizumab as a monotherapy or in combination with other immunomodulators. Also advantageously, the one or more immunotherapeutic agents according to the invention may comprise ipilimumab, nivolumab or ADU-S100 as a monotherapy or in combination with other immunomodulators. Advantageously, the one or more immunotherapeutic agents according to the invention may comprises ipilimumab, pembrolizumab or ADU-S100 as a monotherapy or in combination with other immunomodulators. Again, advantageously, the one or more immunotherapeutic agents according to the invention may comprise ADU-S100, nivolumab or pembrolizumab as a monotherapy or in combination with other immunomodulators.

Advantageously, the one or more immunotherapeutic agents according to the invention may comprise ipilimumab or pembrolizumab as a monotherapy or in combination with other immunomodulators. Advantageously, the one or more immunotherapeutic agents according to the invention comprises ipilimumab or nivolumab as a monotherapy or in combination with other immunomodulators. Advantageously, the one or more immunotherapeutic agents according to the invention may comprise ipilimumab or ADU-S100 as a monotherapy or in combination with other immunomodulators. Advantageously, the one or more immunotherapeutic agents according to the invention may comprise nivolumab or pembrolizumab as a monotherapy or in combination with other immunomodulators. Advantageously, the one or more immunotherapeutic agents according to the invention may comprise nivolumab or ADU-S100 as a monotherapy or in combination with other immunomodulators. Advantageously, the one or more immunotherapeutic agents according to the invention may comprise pembrolizumab or ADU-S100 as a monotherapy or in combination with other immunomodulators.

Preferably, the one or more other immunotherapeutic agents is selected from the group consisting of durvalumab, atezolizumab, tremelimumab, spartalizumab, cemiplimab, pembrolizumab, ADU-S100 and/or nivolumab.

Preferably, the known anti-cancer therapy is the use of olaparib for treatment in cancer. The term “olaparib” relates to olaparib (Lynparza, available from AstraZeneca, Cambridge, UK), a poly ADP ribose polymerase (PARP) inhibitor.

An individual that shows a significant response to an immunotherapeutic treatment is a patient who is affected with a cancer and who will show a clinically significant response after receiving said anticancer treatment; the clinically significant response may be assessed by clinical examination (body weight, general status, pain and palpable mass, if any), biomarkers and imaging studies (ultrasonography, CT scan, PET scan, MRI). According to a specific embodiment, the individual is a patient with ovarian cancer.

“Selection Factor” herein means the effect that the agent shows on the number and size of larger cell aggregates, i.e. cell aggregates larger than 420 μm² in the cell culture; conditioned on statistically significant decrease in abundance of these larger objects and as well conditioned on statistically significant increase in abundance of objects smaller than 160 μm². The increased abundance of smaller objects corresponds to activation of immune cells. For this purpose, the sum of area in all the tumoroids with an area larger than 420 μm² is calculated in each of the multitude of samples, such as preferably in each well of a standard 384-well plate. If this sum of areas is not statistically significantly lower across the replicates in immunomodulator treatment compared to the negative control, then the value of Selection Factor is assigned as zero. The statistical test used here is applied as a one-sided Wilcoxon test, which does not assume normality of the data. A Wilcoxon signed-rank test is a non-parametric statistical hypothesis test used to compare two related samples, matched samples, or repeated measurements on a single sample to assess whether their population mean ranks differ i.e. it is a paired difference test.

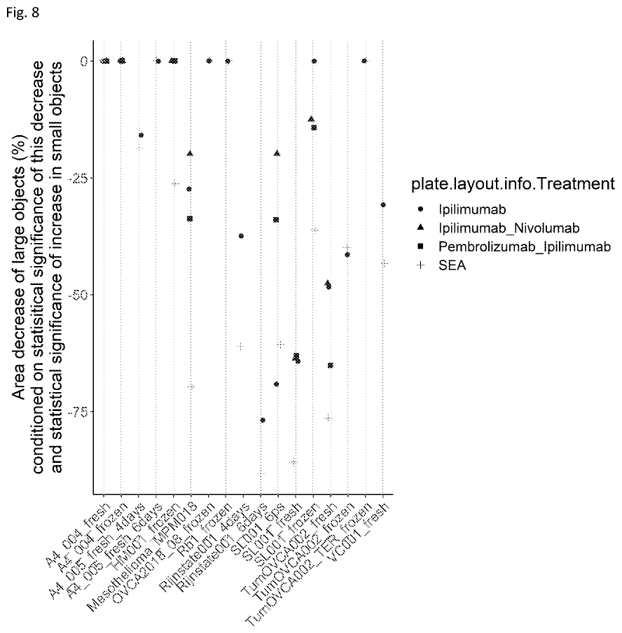

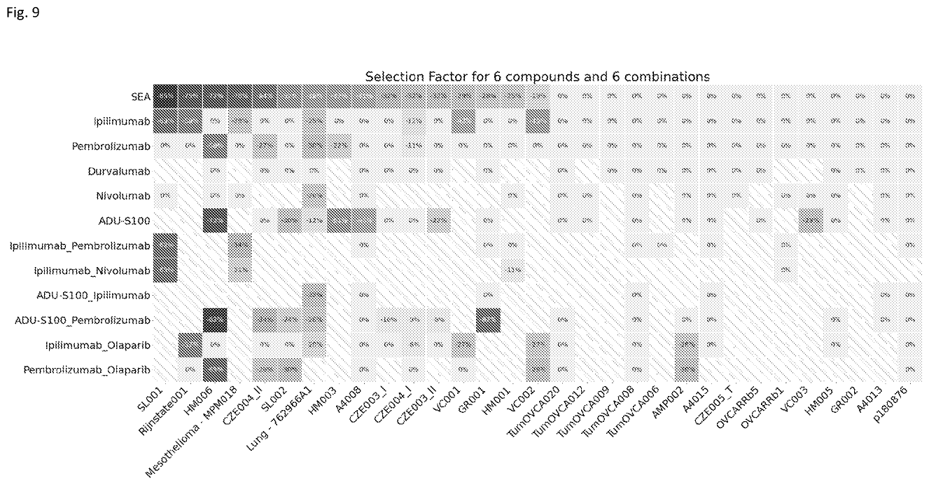

In the next step, it is tested whether there is statistically significant increase in number of objects smaller than 160 μm² in response to the checkpoint inhibitor, such as e.g. Ipilimumab. This next step may also be testing whether there is statistically significant increase in number of objects smaller than 160 μm² in response to pembrolizumab, nivolumab or ADU-S100. For this purpose, the sum of the area of the small objects with an area<160 μm² in each well of the 384-well plate is calculated. If this sum of areas is not statistically significantly higher across the replicates in the checkpoint inhibitor, e.g. Ipilimumab, pembrolizumab, nivolumab or ADU-S100 treatment compared to the negative control, then the Selection Factor is assigned as zero. In case both these two statistical test checks are met, we assign Selection Factor as percentage of decrease in the across-replicate median of decrease in area of tumoroids (large objects). The measured metric across different samples are shown in FIGS. 8 and 9 for ipilimumab, pembrolizumab, nivolumab, ADU-S100, and olaparib, which showed that these compounds were, alone or in combination, able to start an immune reaction that effectively reduced the number of tumour cells, based only on the immune system of a naïve patient sample. Without wishing to be bound by any particular theory, it is believed that the statistically significant increase in number of objects smaller than 160 μm² relates to an increase in immune cells, which typically appear as objects smaller than 160 μm² area.

As used herein, the term “cancer” refers to or describe the physiological condition in mammals in which a population of cells are characterized by unregulated cell growth. Examples of cancer include, but are not limited to, carcinoma, lymphoma, blastoma, sarcoma, and leukaemia. More particular examples of such cancers include metastatic or non-metastatic disease of any types of lung cancer, peritoneal cancer, gastrointestinal cancer, pancreatic cancer, melanoma, glioblastoma, cervical cancer, ovarian cancer, liver cancer, bladder cancer, liver cancer, breast cancer, colon cancer, colorectal cancer, endometrial or uterine carcinoma, salivary gland carcinoma, kidney cancer, liver cancer, prostate cancer, vulvar cancer, thyroid cancer, hepatic carcinoma and head and neck cancer.

The term “treatment” herein refers to any reduction of the progression, severity and/or duration of cancer.

The present invention also encompasses a pharmaceutical composition useful in the treatment of proliferative diseases, more specifically cancer, more specifically ovarian cancer, lung cancer and mesothelioma, yet more specifically ovarian cancer, even more specifically lung cancer, comprising the administration of a therapeutically effective amount of the agents, or agent combinations according to the invention, with or without pharmaceutically acceptable carriers or diluents. The pharmaceutical compositions according to the invention advantageously comprise an anti-proliferative agent or agents, and a pharmaceutically acceptable carrier.

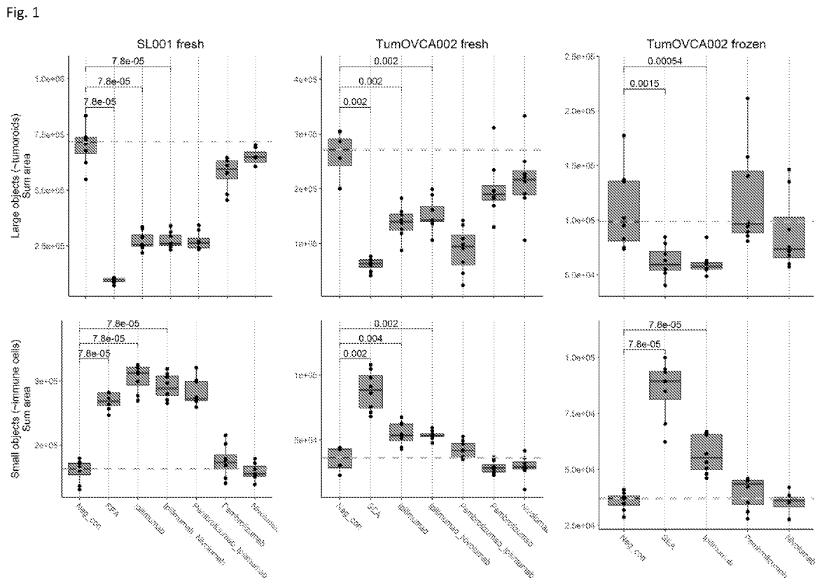

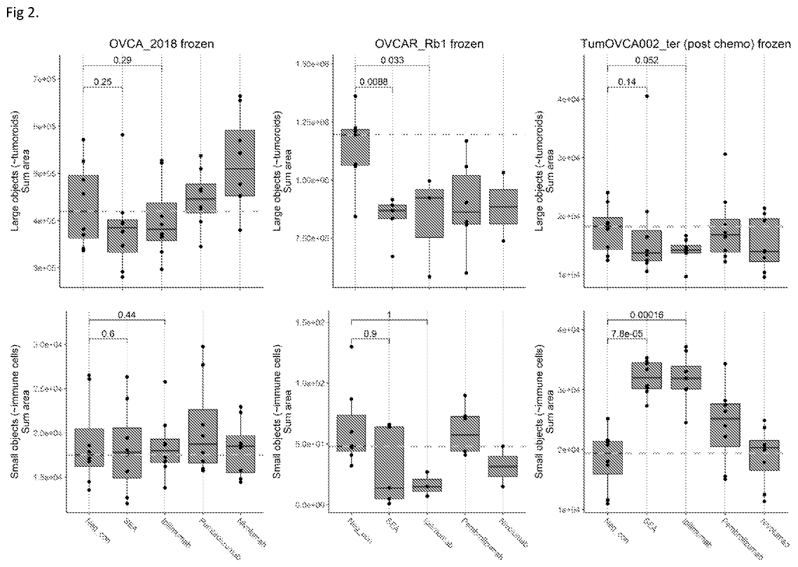

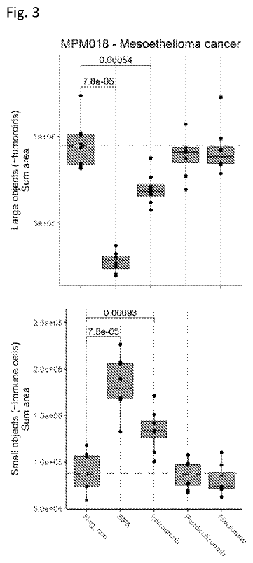

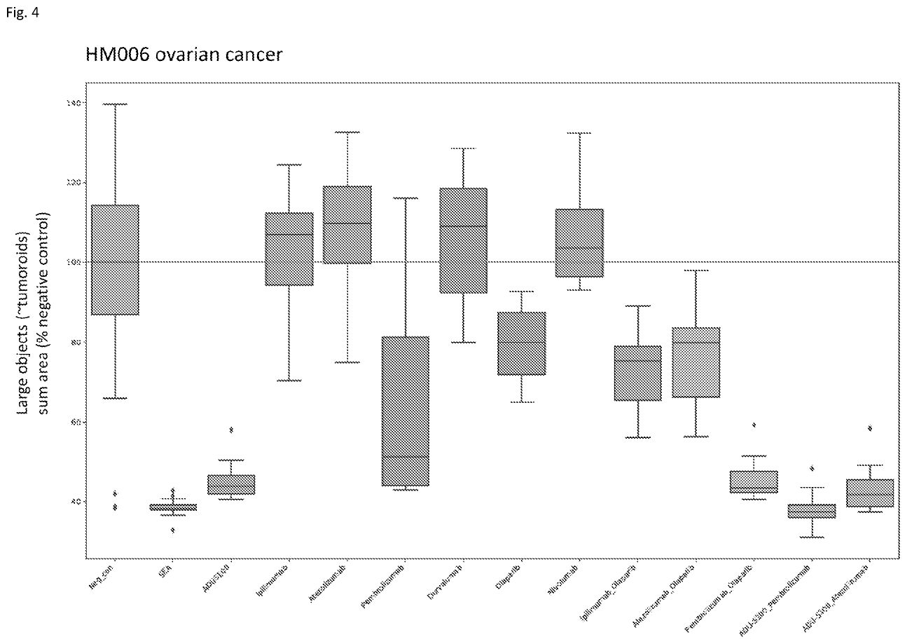

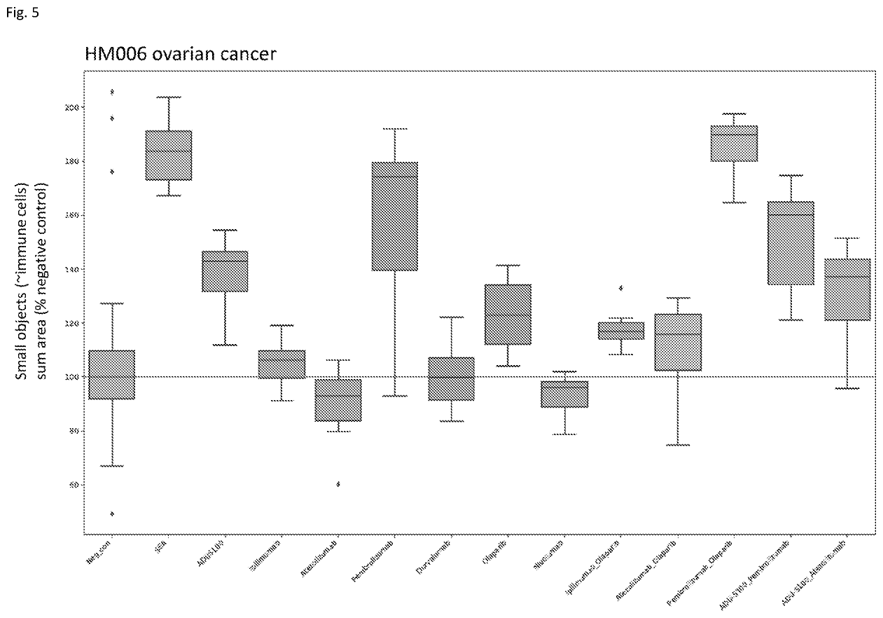

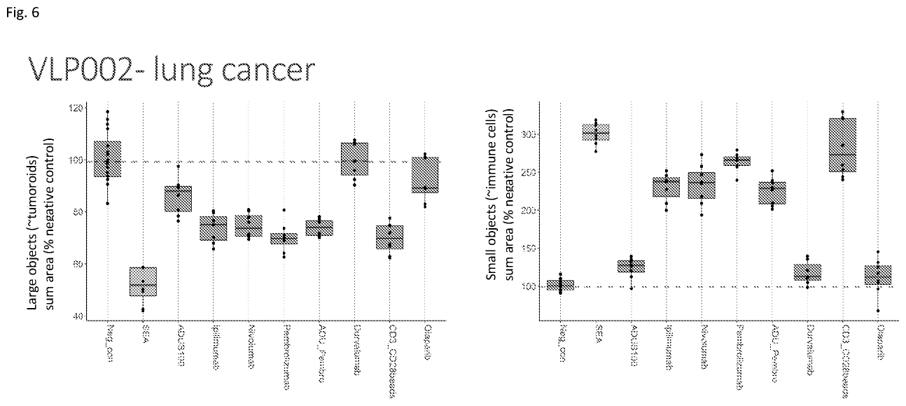

Given the high cost, significant side effects, and patient-to-patient response variability of immunotherapeutic agents for cancer treatment, it is highly appealing to develop an in-vitro assay to predict response of individual patients to these treatments. Applicants have developed an assay which can gauge immunotherapeutic agents for treatment efficiency for a.o. ovarian, lung and mesothelioma cancer patients. The assay can advantageously also be used to measure the immune-mediated effect of one or more immunotherapeutic agents in vitro. This assay is based on ex vivo 3D culturing of individual patient's tumour tissues, i.e. derived from fresh or cryopreserved samples, in combination with 3D imaging, and subsequent morphological characterization of cell types in the sample as set out for instance in Booij, T. H., Bange, H., Leonhard, W. N., Yan, K., Fokkelman, M., Kunnen, S. J., . . . Price, L. S. (2017), High-Throughput Phenotypic Screening of Kinase Inhibitors to Identify Drug Targets for Polycystic Kidney Disease, SLAS DISCOVERY: Advancing Life Sciences R&D, 22(8), 974-984. Representative results from this assay are shown in FIGS. 1-6. For the samples in FIG. 1, Ipilimumab showed effectiveness in activating immune cells for ovarian cancer, in order to kill the tumoroids, while similar effect were not observed in samples shown in FIG. 2. FIG. 3 shows a sample where we see killing effect in a mesothelioma cancer sample. FIG. 4 shows a sample where we see a killing effect, i.e. area size reduction of tumoroid clusters, in an ovarian tumour sample when treated with ADU-S100, and pembrolizumab, and combinations thereof. FIG. 5 shows the same sample of FIG. 4, where we see area size increase of small objects in an ovarian tumour sample when treated with ADU-S100, and pembrolizumab, and combinations thereof. FIG. 6 shows a sample where we see killing effect in a lung cancer tumour sample when treated with ADU-S100, and pembrolizumab, ipilimumab, nivolumab, and combinations thereof.

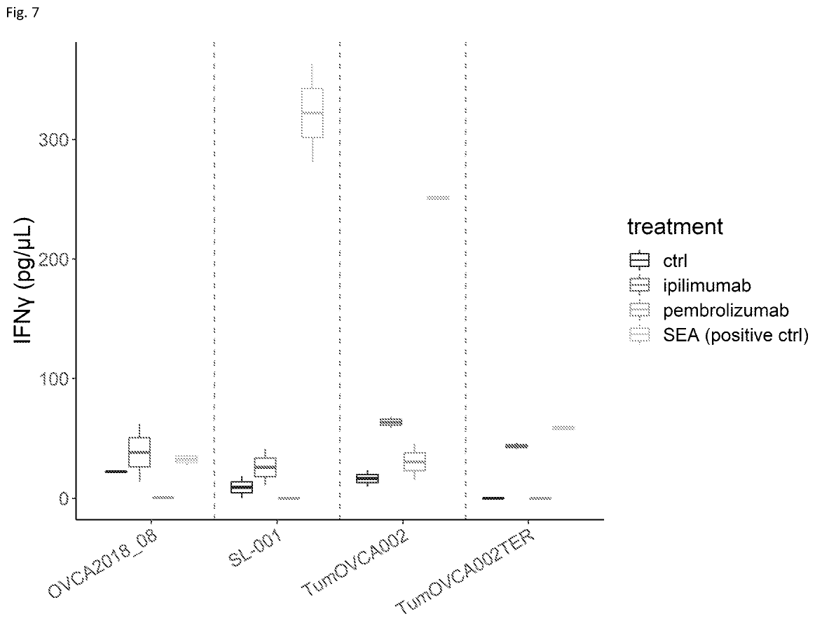

FIG. 7 shows that the observed tumour killing effect is accompanied by IFN-γ production, an evidence for presence of activated T-cells. Based on these observations, applicants found that statistical test-conditioned decrease in tumoroid area of tumoroid clusters larger than approximately 420 μm² was suitable as Selection Factor as a read-out for Ipilimumab efficacy in each patient, thereby effectively allowing for a patient selection.

Similarly, applicants found that statistical test-conditioned decrease in tumoroid area of tumoroid clusters larger than approximately 420 μm² was suitable as Selection Factor as a read-out for pembrolizumab, nivolumab and ADU-S100 efficacy in each patient, thereby effectively allowing for a patient selection.

In the present method, preferably, a multitude of replicates are prepared and analysed in parallel.

Advantageously, the multitude of samples are prepared from the tissue or fluid sample in parallel, wherein each sample is placed in a well on a microtiter plate, and wherein each sample comprises of from 100 to 300 aggregates in a volume of from 1 to 20 μl of a suitable growth matrix, preferably a hydrogel and of from 19 to 40 μl of a suitable medium, wherein the total volume of a sample is 60 μl.

Advantageously, the multitude of samples are prepared from the tissue or fluid sample in parallel, wherein each sample is placed in a well on a microtiter plate, and wherein each sample comprises of from 1 to 800 aggregates, preferably of from 10 to 800 or of from 1 to 500, more preferably of from 10 to 100 or of from 100 to 500, even more preferably of from 200 to 300 aggregates, in a volume of from 1 to 20 μl of a suitable growth matrix, preferably a hydrogel and of from 19 to 40 μl of a suitable medium, wherein the total volume of a sample is 60 μl.

Preferably, step (a) comprises providing a test sample comprising patient-derived tumour cellular material and immune cells from a mammalian tumour tissue or fluid sample by: (i) subjecting the sample to mild shearing and/or filtration to obtain homogenized cellular material isolated cells and cell aggregates ranging from 30-100 μm in diameter (can be variable depending on the starting material); and

(ii) enriching of the sample is achieved by filtration to reduce the number of aggregates larger than 100 μm in diameter, and/or reduce aggregates smaller than 30 μm, advantageously by passing the sample through a mesh filter with the appropriate mesh size;
(iii) embedding the homogenized cellular material with a growth medium for a period and under conditions suitable for three-dimensional cell culture comprising aggregates of a surface area of more than 420 μm²;

Preferably, the tissue sample may be directly employed after sampling and optional transport, or as a sample cryopreserved according to a standard protocol for preserving viability of immune cells present in the sample, or wherein the sample is split into a fresh sample and a cryopreserved sample for correlation of the data at a later point in time. Advantageously, wherein normalizing the average size of the sample contents by subjecting the sample to mild shear and/or filtration to enrich for the preferred size of aggregates ranging from 30-100 μm in diameter in the growth medium. Preferably the shearing is conducted by passing the tumour sample through a syringe needle one or more times, such as at least 3 times through a G25 syringe. The shearing may also be conducted by passing the tumour sample through other types of syringes or through a pipette one or more times, such as at least 3 times. Filtration is performed to by using filters with appropriate mesh size.

Preferably, the samples comprising tumour cells are derived from a from a patient with metastatic or non-metastatic cancer. More particular examples of such cancers include any types of lung cancer, peritoneal cancer, gastrointestinal cancer, pancreatic cancer, melanoma, glioblastoma, cervical cancer, ovarian cancer, liver cancer, bladder cancer, breast cancer, colon cancer, colorectal cancer, endometrial or uterine carcinoma, salivary gland carcinoma, kidney cancer, prostate cancer, vulvar cancer, thyroid cancer, hepatic carcinoma and head and neck cancer, preferably cancer is selected from ovarian cancer, lung cancer and mesothelioma, preferably cancer is selected from ovarian cancer and mesothelioma, preferably cancer is lung cancer. Preferably, the tumour cell culture comprises a naïve sample is derived from resected tumour specimen, tumour biopsies or malignant fluids (e.g. ascites, pleural effusion). Preferably, the three-dimensional culture comprises tumour cells and immune cells. Preferably, the patient derived tumour sample is the only source of immune cells in an ex vivo three-dimensional (3D) patient derived tumour culture. Ex vivo 3D patient derived tumour cultures in the art are commonly analysed on immune-mediated effects of one or more immunotherapeutic agents by adding an additional exogenous source of immune cells and therefore they do not solely rely on the immune cells present in the ex vivo 3D patient derived tumour culture. The ex vivo 3D patient derived tumour cultures preferably do rely only on the native immune cells that are present in the patient derived tumour sample after isolation from a patient.

Preferably, the three-dimensional culture comprising ex vivo tumour aggregates in a hydrogel is prepared by subjecting a tumour sample to shearing and/or filtration, to yield a cell culture comprising cells and cell aggregates ranging from 30-100 μm in diameter, based on the filter exclusion size.

Preferably, the three-dimensional culture comprising ex vivo tumour aggregates in a hydrogel is prepared by subjecting a tumour sample to shearing and/or filtration, to yield a cell culture comprising cells and cell aggregates substantially ranging from 30-100 μm in diameter. A person skilled in the art knows that fully homogenising a tumour sample by shearing and/or filtration is difficult and a standard normal distribution is more likely to be achieved instead, hence the preference to yield a cell culture comprising cells and cell aggregates substantially ranging from 30-100 μm in diameter.

Preferably, the culturing period in step (c) is between about 3 and 7 days. Preferably, the objects that have a surface area of above 420 μm² are tumour cell aggregates or tumoroids and the object that are below about 160 μm² are considered immune cells.

Preferably, prior to the 3D imaging, the cell culture is stained, such as with actin staining reagents like tetramethyl rhodamine(TRITC)-phalloidin. Other staining reagents may be actin-staining reagents like rhodamine-phalloidin or deoxyribonucleic acid (DNA) or cell nucleus staining reagents like 4′,6-diamidino-2-phenylindole (DAPI), or, preferably, Hoechst dyes such as Hoechst 33258.

Preferably, step (d) further comprises assessing the viability and/or size of the tumour cell aggregates of a surface area of more than 420 μm² in the presence or absence of the immunotherapeutic agents and/or anti-proliferation agent tested to create comparative data on viability and/or size of the tumour cell aggregates in presence or in absence of the immunotherapeutic and/or anti-proliferation agent, and relating the data obtained to values indicative of immunotherapeutic and/or anti-proliferation agent activity for reducing/increasing viability and/or size of the tumour cell aggregates.

Preferably, step (e) comprises optical scanning of the cultured sample with an automated computer-controlled multifocal fluorescence microscope.

The method according to the invention further preferably comprises providing the sample in a vessel aligned with and functionally coupled to the automated computer-controlled multifocal microscope; determining volumetric imaging parameters; directing excitation light onto a region of interest in the sample; scanning the fluorescence response light across a first portion of the sample; imaging a plurality of layers of the sample in a first volume of the sample in the region of interest to provide first image data; sectioning the first portion of the sample; scanning the excitation light across a second portion of the sample; imaging a second plurality of layers of the sample in a second volume of the sample to provide second image data; and processing the first image data and the second image data to form a three-dimensional image of the sample.

Advantageously, step (d) comprises measuring the effect of the one or more immunotherapeutic agents on viability and/or size of tumour cell aggregates, the method comprising:

i) staining of the cell culture with a fluorescence marker and measuring the fluorescence intensity to determine the total area of stained objects in the culture that are above 420 μm² and below 160 μm′, and ii) capturing a layered fluorescent image of the stained sample; iii) and measuring the object intensity of the luminescent surface areas in the sample; and iv) determining the luminescent surface areas. Advantageously, two fluorescence markers are used, preferably one marking actin and the other marking the cell nucleus or DNA. Advantageously, the sum of area of all tumoroids with an area larger than 420 μm² in each sample is calculated, and wherein it is determined if the sum of all areas is statistically significantly lower across the replicates comprising the same components compared to the negative control. Advantageously, the tumour-reducing effect is derived by calculating the percentage decrease of tumoroid area as a median of a multitude of parallel tests within each replicate, and the median as calculated across the replicates, wherein the tumoroids are distinguished by an area threshold of 420 μm² according to formula I.

$$\{ \begin{matrix}I ) Wilcoxon test : Does total area of large objects decrease ( p < 0.05 ) in treatment condition compared to the negative control ? \\ ‐ No \to Selection Factor = 0 \\ ( II ) Wilcoxon test : Does total area of small objects increase ( p < 0.05 ) in treatment condition compared to the negative control ? \\ ‐ No \to Selection Factor = 0 \\ If ( I ) \& ( II ) are met \to Selection Factor = 100 ⋆ \frac{median\_replicate ( {\sum }_{area \text{?}}^{Treatment} object area ) - median\_replicate ( {\sum }_{area \text{?}}^{Negative \text{?}} object area )}{median\_replicate ( {\sum }_{area \text{?}}^{Negative \text{?}} object area )} \\ \text{?} \text{indicates text missing or illegible when filed}\end{matrix}$$

Herein, a Selection Factor below −30% indicates an effective treatment, and a patient responsive to the treatment. In this formulation the value of Selection Factor is conditioned by statistical increase in tumoroid area reduction (large objects) and amplification of small objects.

Advantageously, step (d) further segmenting the 3-dimensional culture into layers, capturing images of each layer, and deconvoluting the luminescence images of the layers to showing individual cells and cell aggregates in the culture.

Preferably, a decrease in the total area of objects that are above about 420 μm² and an increase in the total area of objects that are less than about 160 μm² compared to a control indicates that the one or more immunotherapeutic agent(s) is(are) effective at reducing the number of tumour cells.

The following, non-limiting examples serve to illustrate the present invention.

### Examples

The following examples show tumour sensitivity vis-à-vis immunotherapeutic agent treatment in three-dimensional tumour aggregates from patients. The tests were performed by immunotherapeutic agent exposure on fresh and cryopreserved ovarian carcinoma, lung carcinoma and mesothelioma-derived primary tumour cells.

Imaging was accomplished using a position-controlled inverted fluorescence microscope stage to enable automated precise localization of each spot incrementally in conjunction with image capture using a CCD camera. Once images are captured, image analysis software defined the area of interest, subtract background, threshold the image to identify cell aggregates as objects, create a region around these objects, and measure their area. These objects can correspond to tumour cell aggregates or immune cells.

Per experiment group sum of object sizes were also determined. In one embodiment of the invention, these analyses can be carried out with existing computer programs, and in another preferred embodiment, software macros automate these steps.

To test treatment efficacy of immunotherapeutic agents, a panel of different immunotherapeutic and anti-proliferation agents were subjected to a three-dimensional (3-D) multi-parametric assay, whereby the ex vivo patient-derived cell aggregates were cultured in extra-cellular based hydrogels in 384-well plates, and in presence and absence of the tested agents.

The culture methods preserve the complex three-dimensional phenotype that facilitates measurement of changes in morphological phenotypes in a high content screen. The aggregates were grown in the presence of the agents at different concentrations. A known immune-activator compound Staphylococcal enterotoxin A (SEA) was included as positive controls, and untreated cell aggregates as negative controls.

Effects on the cells were captured by staining the samples and by collecting 3D image stacks.

Analysis of the data, retaining spatial information, to generate a set of more than 100 different measured features, including the number, shape, and size of objects, cells and nuclei; sub-populations; cell apoptosis and invasion, resulted in an assessment of the Selection Factor indicating the efficacy of any particular treatment.

Preparation of the Cell Cultures

Resected tumour specimen, tumour biopsies or malignant fluids (e.g. ascites, pleural effusion) comprising tumour and immune cells were homogenized to 30-100 μm maximal diameter by shearing with a 25 G needle or a pipette and/or filtration (can be variable depending on the starting material).

This step comprised using either freshly isolated aggregates or cryopreserved and thawed samples prepared by following a standard protocol for preserving the viability of human cells.

Samples were then transferred to a 384 screening well plate as follows, by contacting an aliquot comprising of from 1 to 800, more preferably of from 10 to 500, and again more preferably of from 200 to 300 cell aggregates with a gel composition comprising 50-70% matrigel, 0.2 to 1.0 mg/ml collagen, 5-15% NaHCO₃ and 10% HEPES 1M, in amount of about 80% to about 20% of cell aliquot in its medium. The gel composition can vary depending on the batches of the components and the starting tumour material.

Then per well, and with 8 wells per agent or agent combination, a gel volume per well was added in an amount of from 12 to 15 microliters, with an addition of about 40 μl of medium on top.

Cell Culture Media

A standard cell culture medium was employed, comprising extra-cellular based hydrogel.

Preparation of 3D Screening Plates

Screenings were performed in 384 well plates that were preferably pre-heated at least overnight in a 37° C. incubator, and filled by a liquid handling robot.

Drug Exposure

Drugs and drug combinations were added 24 hours day after plating of the tumour cell aggregates, at a volume to result in a total of 60 ul. Total drug exposure time ranged from 3 to 7 days. Next the tumour cell aggregates were fixed and stained for analysis.

Image Analysis

Images were captured by an inverted computer-controlled fluorescence microscope. Captured images were stored on a central data server, accessible by the OcellO Ominer™ 3D image analysis platform which allows direct parallel analysis of the 3D image stacks by its distributed computational design. This software analyses the structure of the objects (nuclei and cytoskeleton) detected in each well, and their relative positions.

Upon analysis, the output was checked to detect the quality of the raw images and the analysis method. The per-object measurements the software produced (such as its area) were subsequently aggregated per well and the data was coupled to the plate layout information (cell line, growth factor condition, treatment, etc.).

The above experiments showed that treatment with ipilimumab, pembrolizumab, nivolumab and/or ADU-S100 will only benefit a subset of patients, whereby this subset of patients show an in-vitro Selection Factor of below −30%.

1. A method for measuring the immune-mediated effect of one or more immunotherapeutic agents on ex vivo three-dimensional (3D) patient derived tumour cultures, the method comprising:
(a) preparing a three-dimensional size-normalised tumour culture from a patient derived tumour sample in a multitude of replicates;
(b) adding one or more immunotherapeutic agents to the culture, and
(c) culturing for a pre-defined time period; and
(d) determining the effect that the one or more immunotherapeutic agents has on the tumour cell aggregates by measuring the total area of objects in the culture that are above about 420 μm², and the total area of objects that are below about 160 μm², using three-dimensional imaging of the cell culture, and
(e) identifying the patient that responds to one or more immunotherapeutic agents.

2. (canceled)

3. The method according to claim 1, wherein the multitude of samples are prepared from the tissue or fluid sample in parallel, wherein each sample is placed in a well of a microtiter plate, and wherein each sample comprises a suitable amount of, preferably of from 100 to 300, cell aggregates in a suitable volume, preferably of from 1 to 20 μl, of a suitable growth matrix, preferably a hydrogel and a suitable amount of, preferably 50 μl , of a suitable growth medium.

4. The method according to claim 1, wherein the tissue sample may be directly employed after sampling and optional transport, or as a cryopreserved sample according to a standard protocol for preserving viability of human cells present in the sample, or wherein the sample is split into a fresh sample and a cryopreserved sample for correlation of the data at a later point in time.

5. The method according to claim 1, wherein step (d) comprises measuring the effect of the one or more immunotherapeutic agents on ex vivo patient derived 3D tumour cultures, by:
i) staining of the cell culture with a fluorescence marker and measuring the fluorescence intensity to determine the total area of stained objects in the culture that are above about 420 μm² and below 160 μm², and
ii) capturing a layered fluorescent image of the stained sample;
iii) and measuring the object intensity of the luminescent surface areas in the sample; and
iv) determining the luminescent surface areas.

6. The method according to claim 5, wherein the sum of area of all tumour aggregates with an area of above about 420 μm² in each sample is calculated, and wherein it is determined if the sum of all areas is statistically significantly lower across the replicates comprising the same components; and/or wherein the sum of area of all immune cells with an area smaller than about 160 μm² in each sample is calculated, and wherein it is determined if the sum of all areas is statistically significantly higher across the replicates comprising the same components, compared to the negative control.

7. (canceled)

8. The method according to claim 5, wherein the sum of area of all tumour aggregates with an area of above about 420 μm² in each sample is calculated, and wherein it is determined if the sum of all areas is statistically significantly lower across the replicates comprising the same components and wherein the sum of area of all immune cells with an area smaller than about 160 μm² in each sample is calculated, and wherein it is determined if the sum of all areas is statistically significantly higher across the replicates comprising the same components, compared to the negative control; and wherein the effect on tumour aggregates is derived by calculating the percentage decrease of tumour aggregate area as a median of multitude of parallel tests within each replicate, and the median as calculated across the replicates, wherein the tumour aggregates are distinguished by an area threshold of 420 μm² and immune cells are distinguished by having their area smaller than 160 μm² according to formula I:
$$\{ \begin{matrix}I ) Wilcoxon test : Does total area of large objects decrease ( p < 0.05 ) in treatment condition compared to the negative control ? \\ ‐ No \to Selection Factor = 0 \\ ( II ) Wilcoxon test : Does total area of small objects increase ( p < 0.05 ) in treatment condition compared to the negative control ? \\ ‐ No \to Selection Factor = 0 \\ If ( I ) \& ( II ) are met \to Selection Factor = 100 ⋆ \frac{median\_replicate ( {\sum }_{area \text{?}}^{Treatment} object area ) - median\_replicate ( {\sum }_{area \text{?}}^{Negative \text{?}} object area )}{median\_replicate ( {\sum }_{area \text{?}}^{Negative \text{?}} object area )} \\ \text{?} \text{indicates text missing or illegible when filed}\end{matrix}$$
wherein a Selection Factor below −30% indicates an effective treatment, and a patient responsive to the treatment.

9. The method according claim 1, wherein step (d) further segmenting the 3-dimensional culture into layers, capturing images of each layer, and deconvoluting the luminescence images of the layers to enhance the image contrast and create segmentation masks for individual cells and cell aggregates in the culture.

10. The method according to claim 1, wherein a decrease in the total area of objects that are above about 420 μm² and an increase in the total area of objects that are less than about 160 μm² compared to a control indicates efficacy of one or more immunotherapeutic agents on the tumour cells.

11. The method according to claim 1, wherein normalizing the average size of the sample contents by subjecting the sample to mild shear and filtration to yield a homogenised sample with objects of all of all stainable components in the growth medium ranging from 30-100 μm in diameter.

12. The method according to claim 1, wherein the one or more immunotherapeutic agents comprises ipilimumab, nivolumab, pembrolizumab or ADU-S100.

13. The method according to claim 1, wherein the samples comprising tumour cells are derived from a from a patient with metastatic or non-metastatic cancer, preferably lung cancer, peritoneal cancer, gastrointestinal cancer, pancreatic cancer, melanoma, glioblastoma, cervical cancer, ovarian cancer, liver cancer, bladder cancer, liver cancer, breast cancer, colon cancer, colorectal cancer, endometrial or uterine carcinoma, salivary gland carcinoma, kidney cancer, liver cancer, prostate cancer, vulvar cancer, thyroid cancer, mesothelioma, hepatic carcinoma and head and neck cancer, more preferably ovarian cancer or mesothelioma, even more preferably ovarian cancer, lung cancer or mesothelioma.

14. (canceled)

15. The method according to claim 1, wherein the three-dimensional culture comprises tumour cells and immune cells.

16. The method according to claim 1, wherein the three-dimensional culture comprising tumour cells in a hydrogel is prepared by subjecting a tumour sample to shearing and/or filtration, to yield a cell culture comprising cells and cell aggregates ranging from 30-100 μm in diameter prior to the culture step, preferably wherein the shearing is conducted by passing the tumour sample through an orifice, such as a syringe needle one or more times, preferably by passing it at least 3 times through a G25 syringe needle, and passing the sheared sample through a filter with suitable mesh size.

17. (canceled)

18. The method according to claim 1, wherein the culturing period in step (b) is between about 1, preferably and 7 days.

19. The method according to claim 1, wherein the objects that have a surface area of above about 420 μm² are tumour cell aggregates or tumoroids and the object that are below about 160 μm² are considered immune cells.

20. The method according to claim 1, wherein the immunotherapeutic agent is ipilimumab, nivolumab, pembrolizumab or ADU-S100, as a monotherapy, or ipilimumab, nivolumab, pembrolizumab or ADU-S100 is in combination with one or more other immunotherapeutic agents, preferably wherein the one or more other immunotherapeutic agents is selected from the group consisting of durvalumab, atezolizumab, tremelimumab, spartalizumab, cemiplimab, pembrolizumab, ADU-S100 and/or nivolumab.

21. (canceled)

22. The method according to claim 1, wherein prior to the 3D imaging the cell culture is stained with suitable fluorescence marker, preferably with a marker staining actin, preferably wherein step (c) further comprises assessing the viability and/or size of the tumour cell aggregates of a surface area of more than 420 μm² in the presence or absence of the immunotherapeutic and/or anti-proliferation agent tested to create comparative data on viability and/or size of the cell aggregates in presence or in absence of the immunotherapeutic and/or anti-proliferation agent, and relating the data obtained to values indicative of immunotherapeutic and/or anti-proliferation agent activity for reducing/increasing viability and/or size of the primary cell population, preferably further comprising:
(i) providing the sample in a vessel configured to align with and functionally couple to the automated computer-controlled multifocal microscope;
(ii) determining volumetric imaging parameters;
(iii) directing excitation light onto a region of interest in the sample;
(iv) scanning the fluorescence response light across a first portion of the sample;
(v) imaging a plurality of layers of the sample in a first volume of the sample in the region of interest to provide first image data;
(vi) sectioning the first portion of the sample;
(vii) scanning the excitation light across a second portion of the sample;
(viii) imaging a second plurality of layers of the sample in a second volume of the sample to provide second image data; and
(ix) processing the first image data and the second image data to form a three-dimensional image of the sample.

23. The method according to claim 1, wherein step (a) comprises providing a test sample comprising patient-derived tumour cellular material and immune cells from a mammalian tumour tissue or fluid sample by:
(i) subjecting the sample to a shear sufficient to break up cell aggregates to obtain a homogenized cellular material comprising isolated cells and cell aggregates; and
(ii) filtrating the sample to yield a homogenized cell culture comprising cells and cell aggregates ranging from 30-100 μm in diameter, and
(iii) contacting the homogenized cellular material with a growth medium for a period and under conditions sufficient to produce a multitude of three-dimensional cell culture comprising aggregates of a surface area of more than 420 μm²; and
(iv) adding an aliquot comprising a sufficient number of cells or cell aggregates to a hydrogel.

24. (canceled)

25. (canceled)

26. (canceled)

27. A 3-dimensional cell culture obtainable according to claim 1.

28. A kit comprising the cell culture obtained according to claim 1, and an imaging analysing apparatus.
